(19) United States
(12) Patent Application Publication
(10) Pub. No.: US 2022/0054370 A1
(43) Pub. Date: Feb. 24, 2022

(54) MULTILAYERED CATIONIC LIPOSOME FOR ENHANCING SKIN ABSORPTION AND PREPARATION METHOD THEREFOR

(71) Applicant: COSMAX, INC., Hwaseong-si, Gyeonggi-do (KR)

(72) Inventors: Su Ji KIM, Seongnam-si, Gyeonggi-do (KR); Jun Bae LEE, Seongnam-si, Gyeonggi-do (KR); Sung Yun HONG, Seongnam-si, Gyeonggi-do (KR); Myeong Sam PARK, Seongnam-si, Gyeonggi-do (KR)

(73) Assignee: COSMAX, INC., Hwaseong-si, Gyeonggi-do (KR)

(21) Appl. No.: 17/422,803

(22) PCT Filed: Sep. 22, 2020
(86) PCT No.: PCT/KR2020/012758

§ 371 Date: Jul. 14, 2021

(30) Foreign Application Priority Data
Sep. 24, 2019 (KR) 10-2019-0117490

(51) Int. Cl. A61K 8/14; A61K 8/68; A61K 8/63; A61K 8/67; A61K 8/60; A61K 8/35; A61Q 19/00

(52) U.S. Cl. CPC A61K 8/14; A61K 8/68; A61K 8/63; A61K 8/675; A61K 8/676; A61K 2800/63; A61K 8/355; A61K 8/671; A61K 8/678; A61Q 19/00; A61K 8/606

(57) ABSTRACT

Provided are a multilayered cationic liposome for enhancing skin absorption, a cosmetic composition including the same, and a method of preparing the same.

### TECHNICAL FIELD

This application claims priority benefits from Korean Patent Application No. 10-2019-0117490, filed on Sep. 24, 2019, the entire contents of which are fully incorporated herein by reference.

The present disclosure relates to a multilayered cationic liposome for enhancing skin absorption, a cosmetic composition including the same, and a method of preparing the same.

### BACKGROUND ART

Skin consists of the epidermis, dermis, and subcutaneous fat layer, and as the outermost membrane of the body, it plays an important role in protecting the body from harmful environments and maintaining homeostasis. Among them, the stratum corneum, which is the outermost layer of the epidermis, consists of corneocytes, which are protein components, and intercellular lipids. In particular, intercellular lipids are composed of ceramides, cholesterol, free fatty acids, etc., and play a role as a skin barrier, such as by blocking the percutaneous invasion of harmful substances, maintaining moisture in the skin, etc. However, such a skin barrier function of the stratum corneum becomes an obstacle to effective absorption of various active ingredients.

Liposomes are a widely known representative drug delivery system for enhancing skin absorption of effective substances. Since liposomes are composed of phospholipids, which are in vivo substances, they have high biocompatibility and thus are widely used in cosmetics or pharmaceuticals. In addition, liposomes have a hydrophilic space therein, and have a feature of being capable being loaded with a hydrophobic material between a double-layer or multi-layer structure, which is advantageous for delivery of various effective substances to the skin. Liposomes may be prepared into ethosomes, elastic liposomes, polymer-coated liposomes, cationic liposomes, etc. according to components that form a membrane, and these liposomes are different from each other in the principle of delivering active ingredients to the skin. Among them, cationic liposomes, which are liposomes composed of cationic lipids, are easily accessible to a negatively charged skin surface by electrostatic attraction, and thus enhance absorption of active ingredients into the skin.

Therefore, to increase skin absorption rates of active ingredients, it is necessary to develop cationic liposomes with excellent biocompatibility and high adhesion to the skin. It is also necessary to develop liposomes including intercellular lipid components such as ceramide and cholesterol to increase similarity to the skin.

### DESCRIPTION OF EMBODIMENTS

### Technical Problem

An aspect provides a cationic liposome composition including cationic lipids, ceramide, and cholesterol.

Another aspect provides a cosmetic composition including a cationic liposome including phospholipid layers including cationic lipids, cholesterol, and ceramide; and a loading subject including a water-soluble skin active material or an oil-soluble skin active material, which is loaded inside the phospholipid layers.

Still another aspect provides a method of preparing the cationic liposome composition including cationic lipids, ceramide, and cholesterol.

### Solution to Problem

An aspect provides a cationic liposome composition including cationic lipids, ceramide, and cholesterol.

Another aspect provides a cosmetic composition including a cationic liposome including phospholipid layers including cationic lipids, cholesterol, and ceramide; and a loading subject including a water-soluble skin active material or an oil-soluble skin active material, which is loaded inside the phospholipid layers.

As used herein, the term “liposome” refers to a vesicle with a diameter of about 50 nm to about 2000 nm surrounded by a fine spherical membrane, and is a concept including all compartments surrounded by lipid bilayers.

As used herein, the term “cationic lipid” means a lipid having a positive net charge at selected pH, such as physiological pH, wherein the physiological pH may be 6 to 8, specifically, 6.5 to 8, and more specifically, 7.5.

The cationic lipid may be dimethyldioctadecylammonium bromide (DDA), 1,2-dioleoyl-3-trimethylammonium-propane (DOTAP), 3β-[N—(N′,N′-dimethylaminoethane) carbamoyl cholesterol (DC-Chol), 1,2-dioleoyloxy-3-dimethylammoniumpropane (DODAP), 1,2-di-O-octadecenyl-3-trimethylammonium propane (DOTMA), 1,2-dimyristoleoyl-sn-glycero-3-ethylphosphocholine (14:1 Etyle PC), 1-palmitoyl-2-oleoyl-sn-glycero-3-ethylphosphocholine (16:0-18:1 Ethyl PC), 1,2-dioleoyl-sn-glycero-3-ethylphosphocholine (18:1 Ethyl PC), 1,2-distearoyl-sn-glycero-3-ethylphosphocholin (18:0 Ethyl PC), 1,2-dipalmitoyl-sn-glycero-3-ethylphosphocholine (16:0 Ethyl PC), 1,2-dimyristoyl-sn-glycero-3-ethylphosphocholine (14:0 Ethyl PC), 1,2-dilauroyl-sn-glycero-3-ethylphosphocholin (12:0 Ethyl PC), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dimyristoyl-3-dimethylammonium-propane (14:0 DAP), 1,2-dipalmitoyl-3-dimethylammonium-propane (16:0 DAP), 1,2-distearoyl-3-dimethylammonium-propane (18:0 DAP), N-(4-carboxybenzyl)-N,N-dimethyl-2,3-bis(oleoyloxy)propan-1-aminium (DOBAQ), 1,2-stearoyl-3-trimethylammonium-propane (18:0 TAP), 1,2-dipalmitoyl-3-trimethylammonium-propane (16:0 TA), 1,2-dimyristoyl-3-trimethylammonium-propane (14:0 TAP), N4-Cholesteryl-Spermine (GL67), polyquaternium-10, polyquaternium-7, guar hydroxypropyltrimonium chloride, cocamidopropylamine oxide, stearamidopropyl dimethylamine, or a combination thereof.

As used herein, the term “ceramide” is a kind of sphingolipids known to perform a function of protecting the skin from stress by removing cells damaged due to internal/external stress. Ceramide may be ceramide EOP, ceramide NS, ceramide NP, ceramide AS, ceramide EOS, ceramide AP, ceramide NDS, glucosyl ceramide, omegahydroxy ceramide, or a combination thereof.

As used herein, the term “cholesterol” is a main component constituting the membrane system of cells, together with ceramide and fatty acids, in the structure of the skin keratin, and the cholesterol may be cholesterol, cholesteryl chloride, cholesteryl octanoate, cholesteryl nonanoate, cholesteryl oleyl carbonate, cholesteryl isostearyl carbonate, or a combination thereof.

The cationic liposome composition may have a multilayer structure. The term “multilayer structure” may refer to a structure consisting of three or more layers that separate an inner phase and an outer phase. Since the cationic liposome composition has the multilayer structure, it may exhibit more beneficial effects in terms of loading of an active ingredient and skin permeation of the active ingredient. Specifically, the cationic liposome may have a multilayer structure, in which the water-soluble skin active material is placed between the phospholipid layers, and the oil-soluble skin active material is placed inside the phospholipid layer. As described above, since the skin active materials are separated and stably present in each layer of the multilayer structure, it is possible to stably deliver the skin active materials, such as nutrients, etc., to the dermal layer of the skin without damaging the carrier. In one specific embodiment, structures of the cationic liposome and a general liposome were examined using a transmission electron microscope, and as a result, it was confirmed that the cationic liposome prepared by including 1,2-dioleoyl-3-trimethylammonium propane, cholesterol, and ceramide exhibited a multilayer structure, whereas the general liposome exhibited a bilayer structure.

A weight ratio of the ceramide and cholesterol may be 1 to 10:40 to 60, for example, 1 to 10:45 to 60, for example, 1 to 8:40 to 55, for example, 1 to 6:40 to 55, for example, 1 to 6:45 to 55, for example, 1 to 4:40 to 55, for example, 1 to 4:45 to 55, for example, 1 to 3:40 to 55, for example, 1 to 3:45 to 55. In the weight ratio, when the ratio of ceramide increases, a precipitation phenomenon may occur due to crystallinity, and when the ratio of cholesterol increases, the membrane becomes too rigid, which may cause an adverse problem in releasing active ingredients.

A zeta potential of the cationic liposome may exhibit a positive potential. The zeta potential of the cationic liposome may be, for example, 1 mV to 80 mV, for example, 5 mV to 75 mV, for example, 10 mV to 60 mV, for example, 15 mV to 55 mV, and for example, 20 mV to 50 mV under a neutral pH condition. In one specific embodiment, it was confirmed that the zeta potential of the general liposome without cationic lipid was measured as a negative value, whereas the zeta potential of the cationic liposome prepared by including 1,2-dioleoyl-3-trimethylammonium propane, cholesterol, and ceramide was measured as a positive value.

The cationic liposome composition may be prepared by a method known in the art, and for example, may be prepared by a thin film hydration method. For example, as a hydrating fluid of water-soluble (hydrophilic) materials, an aqueous solution thereof is used, or a drug or a drug solution is added at any stage during the process of preparing liposomes, thereby preparing water-soluble material-entrapped liposomes. In addition, the oil-soluble (hydrophobic) material may be dissolved in an organic solution of the constituent lipids, and then evaporated to form a dried drug-containing lipid film, followed by hydration.

Since the cosmetic composition includes the cationic liposome, the degrees of skin permeation and skin absorption of the active ingredients included in the cosmetic composition may be remarkably increased. In one specific embodiment, niacinamide as an active ingredient was entrapped in the cationic liposome or the general liposome, and skin permeability thereof was compared, and as a result, it was confirmed that the degrees of skin permeation and skin absorption of niacinamide entrapped in the cationic liposome prepared by including 1,2-dioleoyl-3-trimethylammonium propane, cholesterol, and ceramide were significantly increased, as compared with those of the general liposome.

The water-soluble or oil-soluble skin active material may refer to a material that may give a positive effect or action to the skin, and for example, it may exhibit various skin-improving effects including antioxidant, skin whitening, skin barrier strengthening, skin elasticity improvement, skin wrinkle improvement, skin protection from ultraviolet rays, recovery of skin damage caused by ultraviolet rays, skin moisturizing, skin regeneration enhancement, skin inflammation improvement, skin anti-aging, etc.

The water-soluble skin active material may be niacinamide, ascorbic acid, adenosine, a plant extract, or a combination thereof.

The oil-soluble skin active material may be retinol, retinyl acetate, retinyl parmitate, Coenzyme Q10, α-tocopherol, tocopherol acetate, a plant extract, a plant extract essential oil, or a combination thereof.

A formulation of the cosmetic composition is not limited, as long as it is a common cosmetic formulation, but it may be, for example, a face lotion such as a softening lotion, an astringent lotion, a nutrient lotion, etc., a nourishing cream, a massage cream, an essence, an eye cream, an eye essence, a cleansing cream, a cleansing foam, a cleansing water, a pack, a powder, a body lotion, a body cream, a body oil, or a body essence.

The cosmetic composition may further include a preservative, a stabilizer, a surfactant, a solubilizer, a moisturizer, an emollient, an UV absorber, a preservative, a disinfectant, an antioxidant, a pH adjuster, organic and inorganic pigments, a fragrance, a cooling agent, or an anhidrotic agent, etc. A blending amount of the additional ingredients such as the moisturizing agent, etc. may be easily selected by those skilled in the art within the range that does not impair the purpose and effect of the present disclosure.

Still another aspect is to provide a method of preparing the cationic liposome composition including cationic lipids, ceramide, and cholesterol. The cationic lipids, ceramide, cholesterol, cationic liposome composition, etc. are the same as described above.

The method of preparing the cationic liposome composition may include dissolving cationic lipids, ceramide, and cholesterol in an organic solvent to prepare a solution; forming a lipid membrane by removing the solvent from the solution; and drying and hydrating the lipid membrane.

The method may further include homogenizing the dried and hydrated lipid membrane.

The organic solvent may be methanol, ethanol, propanol, isopropanol, butanol, acetone, ether, benzene, chloroform, ethyl acetate, methylene chloride, hexane, cyclohexane, or a combination thereof, but is not particularly limited thereto.

### Advantageous Effects of Disclosure

A cationic liposome composition according to an aspect exhibits a significantly high skin permeation level of an active ingredient included in the liposome, as compared with a general liposome, and thus it may be used for enhancing skin absorption of the active ingredient included in the liposome composition.

A cationic liposome composition according to another aspect may be safely used as a cosmetic composition because liposome membrane stability and skin safety are remarkably improved by including cholesterol and ceramide.

### BRIEF DESCRIPTION OF DRAWINGS

FIG. 1 shows mean particle sizes of a cationic liposome, a general liposome, and a cationic liposome without ceramide and cholesterol, over time;

FIG. 2 shows mean zeta potentials (mV) of a cationic liposome, a general liposome, and a cationic liposome without ceramide and cholesterol, over time;

FIG. 3 shows a graph showing a particle size increase of a cationic liposome including ceramide and cholesterol at a weight ratio of 1:20;

FIG. 4A shows a graph showing a particle size increase of a cationic liposome including ceramide and cholesterol at a weight ratio of 1:30;

FIG. 4B shows an image showing a precipitation phenomenon of a cationic liposome including ceramide and cholesterol at a weight ratio of 1:30;

FIG. 5 shows photographs of a multilayer structure of a cationic liposome, as examined by a transmission electron microscope;

FIG. 6 shows results of an in vitro skin permeation test for examining skin absorption ability of a cationic liposome; and

FIG. 7 shows fluorescence microscope images showing results of a skin permeation test using artificial skin to compare a skin permeation degree for examining skin absorption ability of a cationic liposome.

### BEST MODE

### Mode of Disclosure

Hereinafter, the present disclosure will be described in more detail with reference to exemplary embodiments. However, these exemplary embodiments are only for illustrating the present disclosure, and the scope of the present disclosure is not limited to these exemplary embodiments.

### Example 1. Preparation of Cationic Liposome

A cationic liposome was prepared through a thin film hydration method. L-α-phosphatidylcholine (Egg pc), 1,2-dioleoyl-3-trimethylammonium-propane (DOTAP) which is a cationic lipid, ceramide, and cholesterol were put in a round bottom flask, and dissolved in 20 mL of chloroform-methanol (4:1) until completely dissolved, and then the solvent was completely removed using a rotary evaporator, and a thin film was formed on the flask wall. The formed lipid film was dried under vacuum for 12 hours to completely remove the residual solvent, and then hydrated by adding 10 mL of purified water, and then homogenized for 5 minutes using a probe sonicator. The liposome solution thus obtained was passed through a 0.45 μm filter (Minisart CA 26 mm), and used in the experiment.

### Comparative Example 1. Preparation of General Liposome

A general liposome was prepared using the above composition and method, excluding DOTAP which is a cationic lipid from the above composition. Ceramide and cholesterol used in the cationic liposome and the general liposome were used to improve membrane stability, biosimilarity, and skin safety.

### Comparative Example 2. Preparation of Cationic Liposome without Ceramide and Cholesterol

To evaluate the skin absorption efficacy of the cationic liposome and how ceramide and cholesterol used in the preparation of the liposomes of Examples affect the membrane stability and skin safety of the liposomes, a cationic liposome without ceramide and cholesterol was prepared as Comparative Example 2. A specific preparation method is the same as in Example 1.

Compositions of Example 1 and Comparative Examples 1 and 2 are shown in Table 1 below.

TABLE 1

| | Ingredient | Example | | Comparative | | Comparative | |
|---|---|---|---|---|---|---|---|
| Section | (g, %) | 1 | | Example 1 | | Example 2 | |
| 1 | DOTAP | 0.06 | g | — | | 0.06 | g |
| | | (about 0.05 wt %) | | | | (about 0.07 wt %) | |
| 2 | Egg PC | 0.75 | g | 0.75 | g | 0.75 | g |

| | | (about 0.7 wt %) | | (about 0.7 wt %) | | (about 0.9 wt %) |
|---|---|---|---|---|---|---|
| 3 | Ceramide | 0.005 | g | 0.005 | g | — |
| | | (about 0.004 wt %) | | (about 0.005 wt %) | | |
| 4 | Cholesterol | 0.25 | g | 0.25 | g | — |

| | |
|---|---|
| (about 0.2 wt %) | (about 0.2 wt %) |

When the cationic lipid was 0.1% or more, or Egg PC was 0.5% or less or 1.0% or more in the cationic liposome, haze may occur during storage, resulting in poor stability. In addition, when a weight ratio of ceramide and cholesterol was 1 (w/w) to 10 (w/w): 40 (w/w) to 60 (w/w), the highest membrane stability was observed. When the ratio of ceramide in the above weight ratio increases, precipitation may occur due to crystallinity, and when the ratio of cholesterol increases, the membrane becomes too hard, which may be unfavorable to release of the active ingredient, and thus it is important to maintain the appropriate ratio.

### Comparative Examples 3 to 5. Preparation of Cationic Liposomes According to Changes in Ceramide and Cholesterol Contents

To evaluate the skin absorption efficacy of the cationic liposome and how ceramide and cholesterol used in the preparation of the liposomes of Examples affect the membrane stability and skin safety of the liposomes, liposomes were prepared by varying the contents of ceramide and cholesterol. A specific preparation method is the same as in Example 1.

Compositions of Comparative Examples 3 to 5 are shown in Table 2 below.

TABLE 2

| | | Comparative | Comparative | Comparative |
|---|---|---|---|---|
| Section | Ingredient (g, %) | Example 3 | Example 4 | Example 5 |
| 1 | DOTAP | 0.06 | 0.06 | 0.06 |
| | | (about 0.07 wt %) | (about 0.07 wt %) | (about 0.07 wt %) |
| 2 | Egg PC | 0.75 | 0.75 | 0.75 |
| | | (about 0.9 wt %) | (about 0.9 wt %) | (about 0.9 wt %) |
| 3 | Ceramide | 0.005 | 0.005 | 0.01 |
| | | (about 0.005 wt %) | (about 0.005 wt %) | (about 0.01 wt %) |
| 4 | Cholesterol | 0.1 | 0.5 | 0.3 |
| | | (about 0.1 wt %) | (about 0.5 wt %) | (about 0.3 wt %) |

### Experimental Example 1. Evaluation of Physical Properties of Liposome Particles

1.1 Examination of Particle Size and Zeta Potential

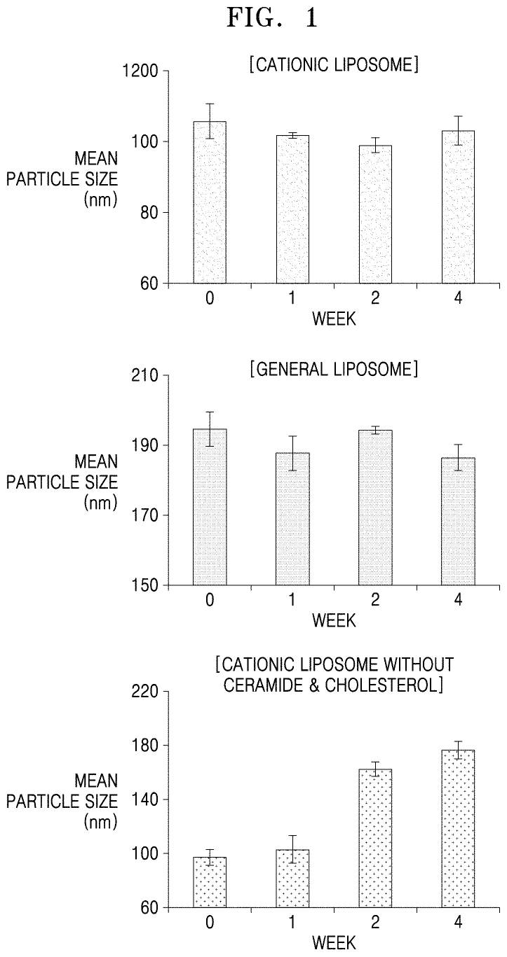

A dynamic light scattering device (DLS, SZ-100, HORIBA) was used under neutral conditions (pH 7) to measure the particle size and zeta potential of the general liposome and the cationic liposome. The results of measuring the particle size and zeta potential for 4 weeks with one-week interval from immediately after preparation are shown in FIGS. 1 to 4A, respectively.

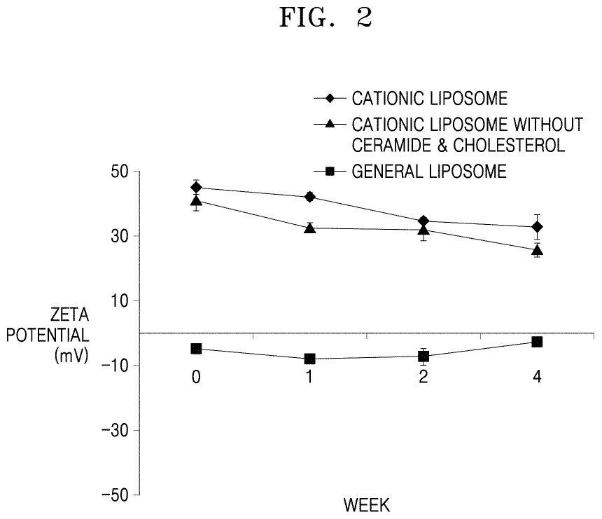

As a result, as shown in FIG. 1, the particle size of the general liposome was measured as 180 nm to 200 nm, and the particle size of the cationic liposome was measured as 100 nm to 120 nm, and as shown in FIG. 2, the zeta potential of the general liposome was −10 mV to 0 mV, and the zeta potential of the cationic liposome was 20 mV to 50 mV, indicating that the surface charge was positive. In other words, stability over time was examined for the particle size and zeta potential of the cationic liposome with cholesterol and ceramide of Example 1, and as a result, it was confirmed that stable physical properties were maintained for 4 weeks. In contrast, the cationic liposome without ceramide and cholesterol of Comparative Example 2 showed that the particle size tended to increase over time. These results confirmed that cholesterol and ceramide play an important role in improving the membrane stability of cationic liposomes.

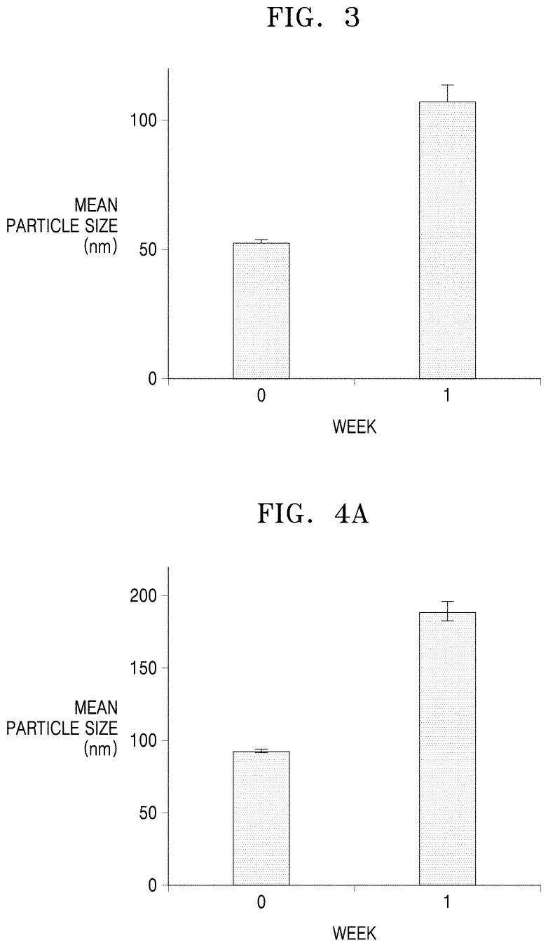

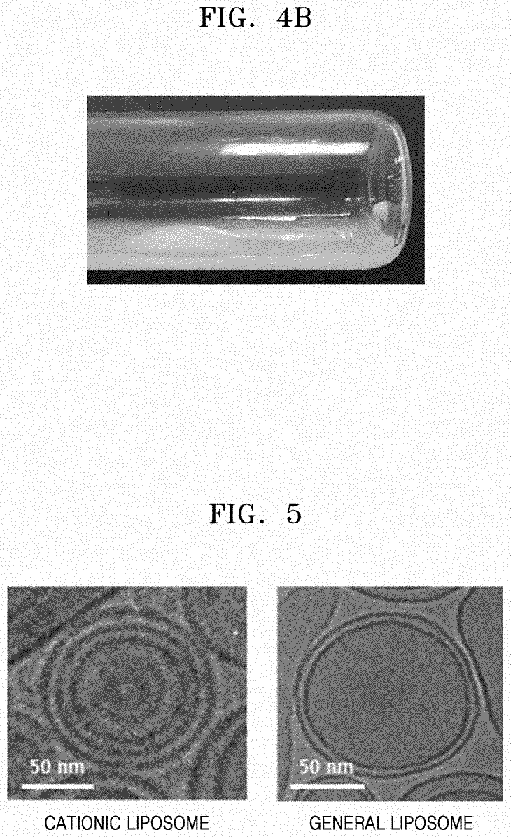

FIG. 3 shows a graph showing the particle size increase of Comparative Example 3, FIG. 4 shows a graph (4A) showing the particle size increase and an image (4B) showing a precipitation phenomenon of Comparative Example 5.

As shown in FIG. 3, the cationic liposome of Comparative Example 3, in which ceramide and cholesterol were included at a weight ratio of 1:20, showed no visible precipitation, but a two-fold increase in the size from about 50 nm to about 100 nm was observed one week after preparation. Further, the cationic liposome of Comparative Example 4, in which ceramide and cholesterol were included at a weight ratio of 1:100, showed a precipitation phenomenon during forming a thin film on the wall of the flask using a rotary evaporator, making it impossible to prepare the liposome. Further, as shown in FIG. 4A, the cationic liposome of Comparative Example 5, in which ceramide and cholesterol were included at a weight ratio of 1:30, showed about two-fold or more increase in the size from about 100 nm to about 200 nm one week after preparation due to the increased content of ceramide, and as shown in FIG. 4B, it was confirmed that the lipid membrane degraded and a precipitation phenomenon occurred. Therefore, when the cationic liposome according to an aspect includes ceramide and cholesterol at a weight ratio of 1 to 10:30 to 60, it may improve the precipitation problem caused by crystallinity and the rigidity problem of the membrane.

1.2 Examination of Particle Structure and Appearance

For structural analysis of the liposome, a cryogenic transmission electron microscopy (cryo-TEM) was used to observe its original structure while maintaining the liposome particle in a cryogenic state. First, 5 μL of liposome was loaded on a 200-mesh carbon lacey film Cu-grid, and then rapidly frozen by immersing the liposome in liquefied (about −170° C.) ethane with a vitrobot. The prepared frozen sample was observed with Cryo-TEM (Tecnai F20, FEI) at an acceleration voltage of 200 kV.

As a result, as shown in FIG. 5, it was confirmed that the cationic liposome formed a multilayer structure which is advantageous for loading of the active ingredient and skin permeation of the active ingredient, and the general liposome formed a bilayer structure.

### Experimental Example 2. In Vitro Skin Permeation Test

To evaluate the skin absorption effect of active ingredient under in vitro conditions for the cationic liposome and the general liposome, each prepared in Example 1 and Comparative Example 1, a skin permeation test was performed using a Franz diffusion cell system. In detail, the general liposome and the cationic liposome, each including niacinamide known as a whitening functional ingredient, were applied in a predetermined amount on an artificial membrane (Strat-M, Merck) for the skin permeation test, respectively, and PBS:EtOH (8:2) was used as a receptor phase. The experiment was conducted at 32° C., and 8 hours after application, the receptor phase was collected through a sampling port, and niacinamide in the collected sample was analyzed using HPLC.

To measure the amount of niacinamide remaining in the stratum corneum and skin after 8 hours, the artificial skin was washed with PBS three times, and then the amount of niacinamide remaining in the stratum corneum was measured using a tape stripping method. The stratum corneum of the skin was peeled off three times using a tape, and put in 10 mL of EtOH, and extracted using an ultrasonic cleaner. After the tape stripping method, the skin from which the stratum corneum was removed was washed and then put in EtOH in the same manner as above, and extracted using an ultrasonic cleaner. Niacinamide in the sample thus obtained was quantified using HPLC. HPLC analysis conditions are shown in Table 3 below. The results of the skin permeation test using the artificial membrane are shown in FIG. 6.

TABLE 3

| | |
|---|---|
| Column | C15 (250 × 4.6 mm, 5 μm, 300 A, Jupiter) |
| Detector | Reversed-phase high performance liquid chromatography |
| | (UltiMate 3000, Dionex) |
| Flow rate | 1.0 mL/min |
| Absorbance | 263 nm |
| Mobile phase | Acetonitrile:Potassium Phosphate monobasic = 3:97 |

The skin absorption effect after 8 hours using the artificial membrane was examined, and as a result, as shown in FIG. 6, the cationic liposome, as compared with the general liposome, showed remarkably increased skin absorption ability in terms of the amount of niacinamide (Tape) present in the stratum corneum, the amount of niacinamide (Membrane) present in the epidermis and dermis, except in the stratum corneum, the amount of niacinamide (Transdermal) permeated through the skin, and the total permeated amount obtained by combining the above amounts.

### Experimental Example 3. Artificial Skin Permeation Test

A skin permeation test was performed using an artificial skin (Neoderm, TEGO SCIENCE) to visually examine the skin permeation degree of the cationic liposome of Example 1, in addition to the results of Experimental Example 2. In detail, 30 μL of fluorescent reagent rhodamine B (Sigma-aldrich)-loaded liposomes were added dropwise to an artificial skin, in which only the epidermal layer existed, and incubated at 37° C. for 2 hours. Thereafter, the support on which the artificial skin was fixed was removed, and the separated artificial skin was put in a mold containing an optimal cutting temperature (OCT) solution, and stored at 80° C. for about 20 minutes, and then sectioned in a size of 20 μm using a cryostat microtome (Leica CM1850, Leica Microsystems). The sectioned tissues were observed with a confocal laser microscopy (LSM-700, Zeiss).

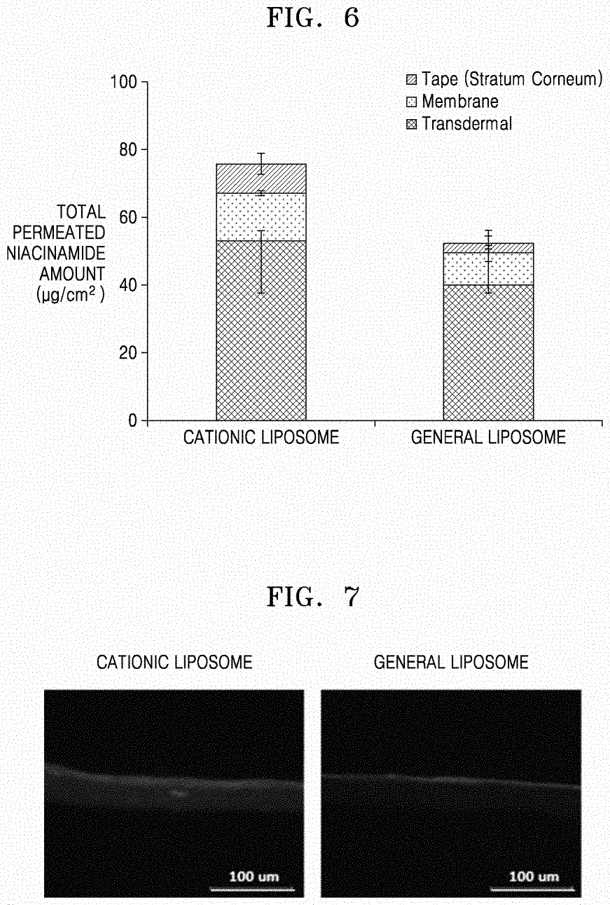

As a result, as shown in FIG. 7, the high fluorescence intensity was observed in the cross section of the skin treated with the cationic liposome of Example 1, as compared with the general liposome, indicating that the cationic liposome penetrated deeper to the lower epidermis. These results are consistent with the results of the in vitro skin permeation test of Experimental Example 2 using the Franz cell diffusion system.

These results are also analyzed such that the fluorescent reagent rhodamine B used in the above experiment, which is a water-soluble fluorescent reagent, is entrapped in the liposome core, and the entrapped fluorescent reagent is highly permeated into the skin, as compared with that of the general liposome, due to affinity of the surface charge of the cationic liposome with the negatively charged skin surface when the liposome particles fuse with the cell membrane and then disperse into the skin cells.

### Experimental Example 4. Skin Safety Test

To compare the skin stability of the cationic liposomes according to inclusion of ceramide and cholesterol, a skin safety test was performed for Example 1 and Comparative Example 2. In detail, skin irritation by the cationic liposomes of Example 1 and Comparative Example 2 was evaluated for 20 male and female adults without skin diseases as follows. After applying 20 μL of the sample to the entire arm of the test subject, the test site was sealed and patched for 24 hours. 30 minutes and 24 hours after removing the patch, the reaction in the skin was examined according to the terminology listed in the CTFA guidelines. The skin irritation index (PII) scores of the test subjects obtained by the criteria were averaged, and if less than 1, it was evaluated as mild irritation, if less than 2, evaluated as slight irritation, if less than 3.5, evaluated as moderate irritation, and if more than 3.5, evaluated as severe irritation.

TABLE 4

| | | Comparative | Comparative |
|---|---|---|---|
| | Example 1 | Example 2 | Example 1 |
| Test | (Cationic | (Cationic | General |
| items | liposome) | liposome) | liposome |
| Skin Irritation | Non-irritation | Mild-irritation | Non-irritation |
| Index (PII) | | | |

As a result, as shown in Table 4 above, the cationic liposome with ceramide and general liposome with ceramide were confirmed to be safely used as a cosmetic composition without irritation, but the cationic liposome without ceramide of Comparative Example 2 showed a skin irritation index of mild-irritation, indicating more irritant. The above results suggest that when the cationic liposome includes ceramide, the skin safety of the liposome may be improved.

Taken together, the above results confirmed that the cationic liposome exhibits a significantly high skin permeation rate of the active ingredient included in the liposome, as compared with the general liposome, and when the cationic liposome includes cholesterol and ceramide, the membrane stability and skin safety of the cationic liposome are remarkably improved.

1. A cationic liposome composition comprising cationic lipids, cholesterol, and ceramide.

2. A cosmetic composition comprising a cationic liposome comprising phospholipid layers comprising cationic lipids, cholesterol, and ceramide; and
a loading subject comprising a water-soluble skin active material or an oil-soluble skin active material, loaded inside the phospholipid layer.

3. The cosmetic composition of claim 2, wherein the cationic lipid is dimethyldioctadecylammonium bromide (DDA), 1,2-dioleoyl-3-trimethylammonium-propane (DOTAP), 3β-[N—(N′,N′-dimethylaminoethane) carbamoyl cholesterol (DC-Chol), 1,2-dioleoyloxy-3-dimethylammoniumpropane (DODAP), 1,2-di-O-octadecenyl-3-trimethylammonium propane (DOTMA), 1,2-dimyristoleoyl-sn-glycero-3-ethylphosphocholine (14:1 Etyle PC), 1-palmitoyl-2-oleoyl-sn-glycero-3-ethylphosphocholine (16:0-18:1 Ethyl PC), 1,2-dioleoyl-sn-glycero-3-ethylphosphocholine (18:1 Ethyl PC), 1,2-distearoyl-sn-glycero-3-ethylphosphocholin (18:0 Ethyl PC), 1,2-dipalmitoyl-sn-glycero-3-ethylphosphocholine (16:0 Ethyl PC), 1,2-dimyristoyl-sn-glycero-3-ethylphosphocholine (14:0 Ethyl PC), 1,2-dilauroyl-sn-glycero-3-ethylphosphocholin (12:0 Ethyl PC), N1-[2-((1S)-1-[(3-aminopropyl)amino]-4-[di(3-amino-propyl)amino]butylcarboxamido)ethyl]-3,4-di[oleyloxy]-benzamide (MVL5), 1,2-dimyristoyl-3-dimethylammonium-propane (14:0 DAP), 1,2-dipalmitoyl-3-dimethylammonium-propane (16:0 DAP), 1,2-distearoyl-3-dimethylammonium-propane (18:0 DAP), N-(4-carboxybenzyl)-N,N-dimethyl-2,3-bis(oleoyloxy)propan-1-aminium (DOBAQ), 1,2-stearoyl-3-trimethylammonium-propane (18:0 TAP), 1,2-dipalmitoyl-3-trimethylammonium-propane (16:0 TA), 1,2-dimyristoyl-3-trimethylammonium-propane (14:0 TAP), N4-Cholesteryl-Spermine (GL67), polyquaternium-10, polyquaternium-7, guar hydroxypropyltrimonium chloride, cocamidopropylamine oxide, stearamidopropyl dimethylamine, or a combination thereof.

4. The cosmetic composition of claim 2, wherein the ceramide is ceramide EOP, ceramide NS, ceramide NP, ceramide AS, ceramide EOS, ceramide AP, ceramide NDS, glucosyl ceramide, omegahydroxy ceramide, or a combination thereof.

5. The cosmetic composition of claim 2, wherein the cholesterol is cholesterol, cholesteryl chloride, cholesteryl octanoate, cholesteryl nonanoate, cholesteryl oleyl carbonate, cholesteryl isostearyl carbonate, or a combination thereof.

6. The cosmetic composition of claim 2, wherein the ceramide and the cholesterol are comprised at a weight ratio of 1 to 10:40 to 60.

7. The cosmetic composition of claim 2, wherein the cationic liposome has a multilayer structure.

8. The cosmetic composition of claim 7, wherein the cationic liposome has a multilayer structure, in which the water-soluble skin active material is located between the phospholipid layers, and the oil-soluble skin active material is located inside the phospholipid layer.

9. The cosmetic composition of claim 2, wherein a zeta potential of the cationic liposome is 10 mV to 60 mV.

10. The cosmetic composition of claim 2, wherein the water-soluble skin active material is niacinamide, ascorbic acid, adenosine, a plant extract, or a combination thereof.

11. The cosmetic composition of claim 2, wherein the oil-soluble skin active material is retinol, retinyl acetate, retinyl parmitate, Coenzyme Q10, α-tocopherol, tocopherol acetate, a plant extract, a plant extract essential oil, or a combination thereof.

12. A method of preparing a cationic liposome composition, the method comprising: dissolving cationic lipids, ceramide, and cholesterol in an organic solvent to prepare a solution;
forming a lipid membrane by removing the solvent from the solution; and
drying and hydrating the lipid membrane.
